(19) United States
(12) Patent Application Publication
(10) Pub. No.: US 2023/0181192 A1
(43) Pub. Date: Jun. 15, 2023

(54) INTRAUTERINE BALLOON WITH EXPANDABLE SUPPORT AND METHODS OF USE

(71) Applicant: Davol Inc., Warwick, RI (US)

(72) Inventors: Maleeha Mashiatulla, Glen Ellyn, IL (US); Amelia Smith, Rochester, MI (US); Melanie Strimas, Mundelein, IL (US); Scott Andras, Little Elm, TX (US)

(21) Appl. No.: 17/549,300

(22) Filed: Dec. 13, 2021

(51) Int. Cl. A61B 17/12; A61N 1/36

(52) U.S. Cl. CPC A61B 17/12136; A61B 17/12099; A61N 1/36007; A61B 2017/4216

(57) ABSTRACT

Hemostasis systems and related methods are generally described. In some embodiments, a hemostasis system may include an inflatable intrauterine balloon. The balloon may be configured to apply pressure to a patient's uterus to reduce the risk of postpartum hemorrhage. In some embodiments, the balloon may be inserted at least partially into the uterus and inflated. A support, which may be located inside the balloon, may expand from a contracted configuration to an expanded configuration to help maintain the balloon's position relative to the cervix when the balloon is inflated. In some embodiments, the support may contract with and/or due to deflation of the balloon, allowing the balloon to move out of the uterus. The balloon may be covered with a hemostatic covering (e.g., gauze preloaded with a hemostatic agent such as hexatomic acid) to enhance the balloon's hemostasis properties.

### FIELD

Disclosed embodiments are related to uterine balloons and related methods of use. More specifically, methods and apparatuses related to uterine balloons with an expandable support for hemostasis are disclosed.

### BACKGROUND

Postpartum hemorrhage is one of the top five causes of maternal mortality worldwide, and can be caused by a variety of conditions such as uterine atony (ineffective contraction of the uterus after delivery), uterine tearing, and improper placenta delivery. Symptoms of postpartum hemorrhage include uncontrolled uterine bleeding, decreased blood pressure, increased heart rates, decrease in red blood cell count, hypotension, and in severe instances, death.

### SUMMARY

In some aspects, medical devices are provided. In some embodiments, a medical device may include a balloon configured to be positioned at least partially in a uterus of a patient. A support may be disposed inside the balloon. The support may have a contracted configuration and an expanded configuration. The support may be configured to expand from the contracted configuration to the expanded configuration due to inflation of the balloon. In the contracted configuration, the support may permit movement of the balloon through a cervix of the patient. In the expanded configuration, the support may inhibit movement of the balloon through the cervix.

In some aspects, methods of operating a medical device are provided. In some embodiments, a method of operating a medical device may include inserting a balloon at least partially in a uterus of a patient, inflating the balloon to apply pressure to the uterus, and expanding a support disposed inside the balloon from a contracted configuration to an expanded configuration. In the expanded configuration, the support may be larger than a cervix of the patient. In the expanded configuration, the support may be positioned in the uterus, superior to the cervix of the patient.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 schematically illustrates a hemostasis system according to some embodiments;

FIG. 2 schematically illustrates an inflated uterine balloon according to some embodiments;

FIG. 3A schematically illustrates a first linkage of a support according to some embodiments;

FIG. 3B schematically illustrates a second linkage of a support according to some embodiments;

FIG. 4A schematically illustrates a front view of first and second linkages of a support in a contracted configuration according to one embodiment;

FIG. 4B schematically illustrates a top view of the linkages of FIG. 4A;

FIG. 4C schematically illustrates a front view of first and second linkages of a support in an expanded state;

FIG. 4D schematically illustrates a top view of the linkages of FIG. 4C;

FIG. 5 schematically illustrates the uterine balloon of FIG. 2 in a deflated configuration according to some embodiments;

FIG. 6 schematically illustrates an inflated uterine balloon according to some embodiments;

FIG. 7 schematically illustrates an expandable linkage according to some embodiments;

FIG. 8A schematically illustrates a front view of linkages of a support in a contracted configuration according to another embodiment;

FIG. 8B schematically illustrates a top view of the linkages of FIG. 8A;

FIG. 8C schematically illustrates a front view of the linkages of a support in an expanded state;

FIG. 8D schematically illustrates a top view of the linkages of FIG. 8C;

FIG. 9 schematically illustrates the uterine balloon of FIG. 6 in a deflated configuration according to some embodiments;

FIG. 10 schematically illustrates an inflated uterine balloon with a three-dimensional support according to some embodiments; and

FIG. 11 schematically illustrates the uterine balloon of FIG. 10 in a deflated configuration according to some embodiments.

### DETAILED DESCRIPTION

Conventional treatment for postpartum hemorrhaging can include medicine or physical massages to induce uterine contraction. In some instances, a flexible vessel, such as a balloon, may be inserted into the uterus and inflated to apply pressure to the uterine walls. The balloon may serve as an intrauterine tamponade, such that the pressure applied to the uterine walls may stop or limit bleeding to achieve hemostasis.

In some cases, the balloon may be used along with absorbent gauze, which may be preloaded with a hemostatic agent. The gauze may be manually packed in the uterus prior to balloon insertion, and may therefore need to be removed from the uterus after use to reduce the risk of infection or adhesion formation.

The inventors have recognized that conventional hemostatic balloons may be difficult to hold in place in the cervix and/or uterus. Due to the excessive flow of fluids and tissue in the area, the balloon (which may be formed of a smooth elastic material) may slip from its position and stop its tamponading action, which may delay hemostasis, reducing the effectiveness of the balloon. A clinician may therefore be required to readjust the intrauterine balloon inside the cervix or uterus of a patient who may be experiencing significant pain and discomfort from a recent delivery.

Based on the foregoing, the inventors have recognized the benefits associated with a hemostasis system for reducing the risk of postpartum hemorrhage. The hemostasis system may be easy to use and may require minimal assembly steps, such that a state of uterine hemostasis may be achieved rapidly, with limited discomfort to the patient.

In some embodiments, a hemostasis system may include an intrauterine balloon, hereinafter referred to as a uterine balloon, configured to be inserted into the uterus of a postpartum patient. The balloon may be inflated to apply a tamponading pressure on the inner walls of the uterus, inducing contraction, which may initiate the hemostasis process. In some embodiments, the use of the uterine balloon may reduce the risk of postpartum hemorrhage. As will be described in greater detail below with reference to the figures, the uterine balloon may include a support which may help to maintain the position of the balloon with respect to the cervix, to reduce the risk of balloon slippage. In some instances, movement of the balloon relative to the uterus may result in a reduced pressure applied to the uterus, which may reduce the efficacy of the uterine balloon in inducing hemostasis.

The uterine balloon may be covered with one or more hemostatic coverings to enhance the hemostasis effect of the balloon. In some embodiments, a hemostatic covering may include one or more segments of gauze (and/or any other suitable absorbent material), which may be pre-loaded with a hemostatic agent. The balloon may be covered with the one or more segments of gauze (and/or any other suitable absorbent material). The gauze may be fixed to the balloon for ease of use. In this way, the balloon and hemostatic loaded gauze may be loaded into (and unloaded from) the uterus in one motion. The gauze may be fixed to the balloon in any suitable manner, such as with a biocompatible adhesive. Of course, other means of attachment between the balloon and gauze are also contemplated, as the present disclosure is not so limited. In some embodiments, a hemostatic covering may include a coating in the form of a hemostatic agent that is directly coated on the balloon, without any gauze or absorbent materials.

In some embodiments, the balloon may be delivered to the patient with a catheter, although other embodiments of balloon delivery are also contemplated. Once the balloon is determined to be at the appropriate position, it may be inflated to apply pressure to the uterine walls. Accordingly, the expanding balloon walls may bring the hemostatic coverings, which may be positioned on an outer surface of the balloon, closer to the uterine walls, and if the balloon pressure is appropriately calibrated, in contact with the walls. In this way, the hemostatic coverings may serve to enhance the hemostasis behavior of the balloon chemically. Of course, embodiments in which the uterine balloon is used independently, without the hemostatic coverings, are also contemplated.

The hemostatic agent included in the coverings may be any hemostatic agent (and/or coagulation agent) known in the art, including, but not limited to, tranexamic acid, calcium chloride, thrombin, glucosamine, chitosan, kaolin, fibrinogen/fibrin, chitin, cellulose, gelatin, alginate, protamine sulfate, and/or any other suitable agent or combination of agents. It should be appreciated that in some embodiments, the hemostatic agent may be delivered to the uterine cavity along with a secondary active material, including, but not limited to, antibacterial, antifungal, anti-inflammatory, analgesics, antihistamines, antibiotics, and/or any other active material or combination of materials.

In some embodiments, the uterine balloon may be inflated with a suitable fluid (e.g., air, water, saline, etc.) via a pump. The pump, which may be a one-way pump in some embodiments, may be connected to a fluid source (e.g., saline bag) via a Luer lock or comparable fluid attachment means. In embodiments where the pump is used to inflate the balloon with air, a filter may be optionally used (e.g., a 200 nm filter) for sterilization purposes. The pump may be operated by one hand, allowing the balloon to be inflated rapidly, requiring limited skill from the clinician. Of course, other methods of uterine balloon inflation known in the art may also be employed, as the present disclosure is not so limited.

The uterine balloon may be formed of any suitable flexible material which may be biocompatible and/or inert. The balloon may be formed of urethane, silicone, natural and/or synthetic rubber, vinyl, neoprene, and/or any other material or combination of materials. Any suitable uterine balloon material known in the art may be employed, as the present disclosure is not so limited. The uterine balloon may also have any suitable fluid capacity (e.g., at least 1000 cc, at least 2000 cc, at least 4000 cc, at least 6000 cc) to sufficiently pack and apply pressure to the uterine walls.

As will be described in greater detail below with respect to the figures, in some embodiments, the hemostasis system may include a support to help maintain the position of the uterine balloon in the uterus. In some embodiments, the support may be positioned inside the balloon. The balloon may be delivered to the uterus (e.g., via a catheter and/or any other suitable means) in a deflated state. In such a state, the support may be in a contracted configuration. In some embodiments, in the contracted configuration, the support may be in a collapsed form. For example, in some embodiments, the support may include a ring of linkages, which may be unlinked in their contracted configuration. In this way, the contracted support may permit movement of the balloon through the cervix and/or uterus. Upon inflation of the balloon (in order to apply pressure to the uterine walls), the support may be urged into an expanded configuration. In some embodiments, in the expanded configuration, the support may have a larger size, such as a larger diameter, than a cervix of the patient. In some embodiments, in the expanded configuration, the support may be positioned in the uterus, superior to the cervix of the patient. As a result, the support may be unable to pass through the cervix toward the vagina, which may help to maintain the position of the balloon inside the uterus. It should be appreciated that in some embodiments, the support may be operated by the inflation/deflation state of the balloon, eliminating the need for a clinician to manually reach inside the patient's vagina or cervix to expand and/or contract the balloon in place.

In embodiments where the support is formed of a ring of linkages, the linkages may substantially engage with one another in the expanded configuration, such that they may form a ring or other assembled structure. The linkages of the support may be designed to form a ring which may be larger than the cervix, such that movement of the balloon out of the uterus may be impeded. In some embodiments, the ring or other assembled structure may be automatically disassembled via the deflation of the balloon. In this way, once a clinician chooses to deflate the uterine balloon (e.g., if a state of hemostasis has been observed), the support may be contracted, and the deflated balloon and support may be comfortably extracted from the patient.

While a ring of linkages is used as an example and shown in the figures, it should be appreciated that the support may adopt any suitable conformation in the contracted and expanded states. For example, as will be described in greater detail below, in some embodiments, the support may have a spherical or ellipsoid shape when in the expanded state. As such, instead of expanding and contracting along two dimensions as a ring would, the support may expand and contract along three dimensions, e.g., as is the case with a spherical or ellipsoid shape.

In some embodiments, the hemostasis system may include one or more electrodes positioned on and/or inside the uterine balloon. The electrodes may be configured to provide electrostimulation to the uterus, which may induce contractions. In this way, the hemostasis properties of the uterine balloon may be enhanced. It should be appreciated that any suitable electrostimulation arrangement and configuration known in the art may be employed with the hemostasis systems of the present disclosure.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

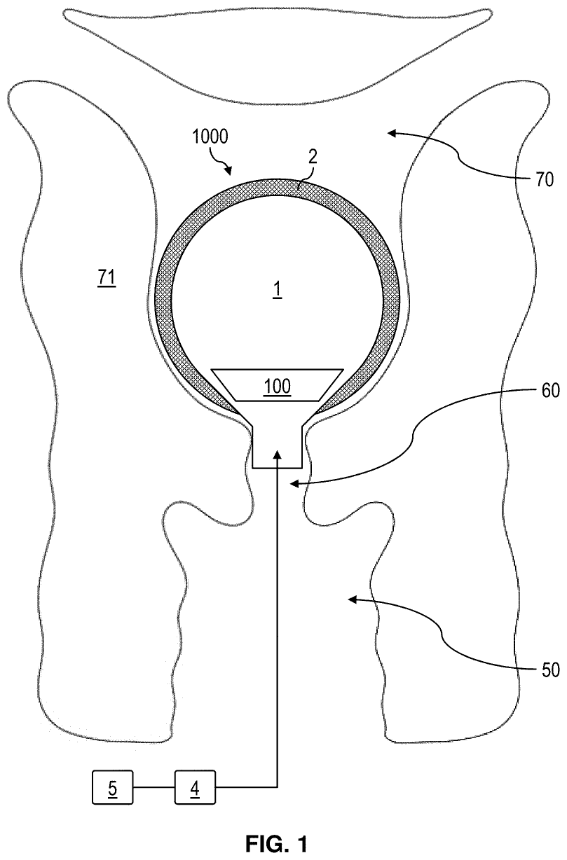

FIG. 1 shows a hemostasis system 1000 deployed in a patient's uterus 70 according to some embodiments. The system 1000 may include at least one uterine balloon 1 configured to be inserted at least partially in the uterus 70. In some embodiments, the balloon 1 may exert pressure on the uterine walls 71 to reduce the risk of hemorrhage. The balloon 1 may be covered with one or more hemostatic coverings 2. The hemostatic covering 2 may be one or more segments of gauze preloaded with a hemostatic agent (e.g., tranexamic acid), or may be a coating of a hemostatic agent applied directly to the balloon, or any other suitable arrangement. Upon inflation of the balloon 1, the hemostatic coverings 2 may be pressed against the uterine walls 71 to assist in the hemostasis process initiated by the balloon pressure. Of course, embodiments of uterine balloon without the hemostatic covering are also contemplated. In some embodiments, the balloon 1 may be inserted into the patient through the vagina 50, positioned past the cervix 60, and subsequently inflated with a pump 5 (e.g., a one-way pump), which may serve to facilitate the flow of fluid from a fluid source 4 to the balloon.

In some embodiments, the balloon 1 may include a support 100, which may help to maintain the position of the balloon 1 with respect to the uterus 70. The support 100 may be driven by the inflation of the balloon, such that the support may help to maintain the position of the balloon when the balloon is inflated, and release the position of the balloon when deflated. As will be described in greater detail below, the support may form an expanded configuration, such as a ring of linkages, which may have a greater diameter or otherwise an otherwise larger size than the cervix 60 when assembled, reducing the risk of the balloon slipping out during the hemostasis process. As shown in FIG. 1, in the expanded configuration, the support 100 may be positioned in the uterus, superior to the cervix 60. The linkages may be unlinked or otherwise disassembled by the deflation of the balloon, releasing the balloon from its position, such that a clinician may extract the balloon 1 from the patient without significant discomfort. In some embodiments, the support 100 may be positioned inside the balloon, such that the support does not directly contact the internal anatomy of the vagina 50, cervix 60, or uterus 70.

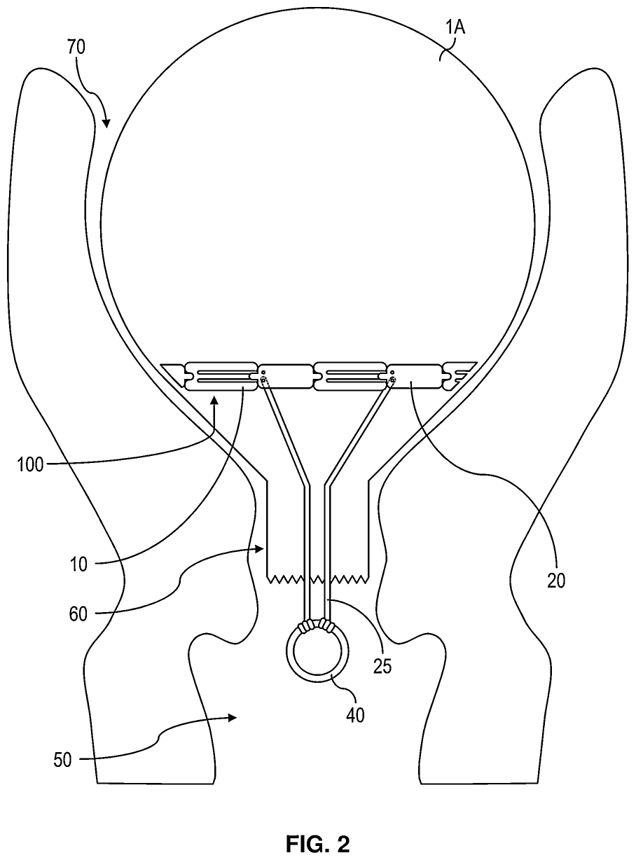

FIG. 2 shows a uterine balloon in an inflated configuration 1A positioned in a uterus 70. The balloon may include a support 100 positioned inside the balloon, which may help to maintain the position of the balloon inside the uterus 70, relative to the cervix 60. As shown in FIG. 2, in the expanded configuration, the support 100 may be positioned in the uterus 70, superior to the cervix 60. In some embodiments, the support 100 may include a set of linkages 10, 20 which may be assembled concurrently with the inflation of the balloon.

In some embodiments, as shown in the figure, the support 100 may be connected to one or more tethers 25 and a pull ring 40. The tethers 25 may allow the support (which may be located above the cervical opening) to be manipulated by a clinician external to the patient. As will be described in greater detail below, the pull ring 40 or any other suitable mechanism may allow the clinician to externally disassemble the support and allow the balloon to be comfortably extracted from the patient's uterus. For example, in some embodiments, the pull ring 40 may be accessible from a patient's vagina 50 or vaginal opening to allow the clinician to operate the balloon from outside the patient.

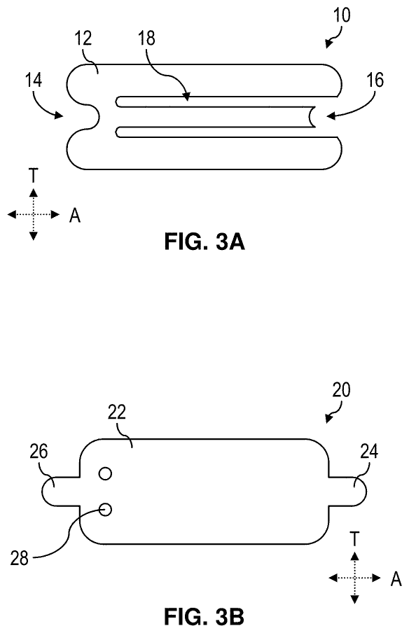

FIGS. 3A-3B depict complementary linkages 10, 20 as part of a support according to some embodiments. FIG. 3A shows a first linkage 10, which may include one or more slots 18 formed in a body 12. The slots 18 may extend at least partially or entirely through a thickness of the body (shown as the in-plane direction of the figure), and generally along the axial direction A of the body 12, as shown in FIG. 3A. In some embodiments, the body 12 may also include a notch or indent 14 at one end of the linkage 10 to accept a portion of the complementary second linkage 20. On an opposing end of the body 12, as shown in the figure, the linkage 10 may include another indent 16, which may be open to the slots 18. In other words, the slots may extend through to the indent 16, such that a feature of second linkage 20 may move from the indent 16 directly into the slots 18.

FIG. 3B shows the complementary second linkage 20 according to some embodiments. The linkage 20 may include at least one protrusion 24 which may be sized to fit into indent 14 of first linkage 10. The protrusion 24 may extend from a first end of the body 22 of the second linkage 20. At an opposing end, the body 22 may include a second protrusion 26. The second protrusion 26 may be sized to fit into indent 16 of the linkage 10. In some embodiments, the body 22, first protrusion 24, and second protrusion 26 may have the same in-plane thickness. The second linkage 20 may also include one or more protrusions 28 extending from the plane of the body 22. For sake of clarity, the protrusions 28 will be hereinafter referred to as “pegs” to prevent any confusion with protrusions 24 and 26, extending along the plane of the body 22. It should be appreciated that the term “peg” is not limited to any particular structure, shape, and/or size, and may describe any body extending out of plane of the second linkage body 22.

In some embodiments, pegs 28 may be positioned proximal to the second protrusion 26. The pegs 28 may be sized to fit within and slide along the slots 18 along the axial direction A. In some embodiments, as shown in FIGS. 3A-3B, two slots 18 may correspond to two pegs 28. The presence of more than one slot/peg pairing may serve to stabilize the linkages 10, 20 along the transverse direction T. However, the present disclosure is not limited by the number of and/or arrangement of slots and pegs.

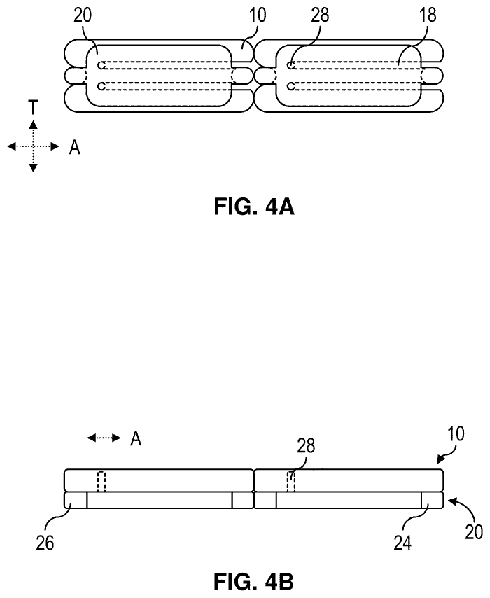

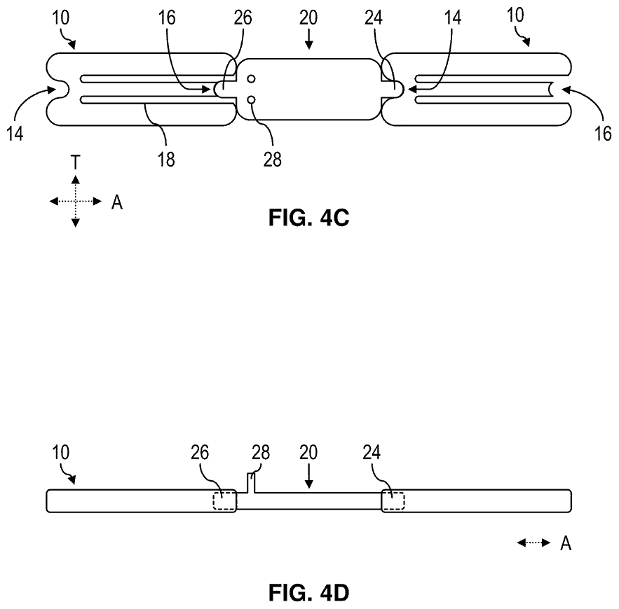

FIGS. 4A-4D depict a process of expanding the support (see support 100 in FIG. 2) according to some embodiments. It should be appreciated that FIGS. 4A-4D depict a partial view of the support (e.g., only a few linkages), and that any suitable number of linkages may be employed for the support, as the present disclosure is not so limited.

FIGS. 4A-4B depict a set of first linkages 10 and second linkages 20 overlapping with one another in a contracted configuration. In this configuration, the linkages 10, 20 may be engaged with one another via the pegs 28 inside slots 18, as shown in the front view of FIG. 4A and the top view of FIG. 4B. In this way, the second linkages 20 may be free to move relative to the first linkages 10 along the axial direction A along the slots 18. In some embodiments, the second linkage 20 may be coupled to the first linkage 10 in the transverse direction T through the engagement of the pegs 28 and slots 18. It should be appreciated that although neighboring linkages are shown to be in contact with one another in FIGS. 4A-4B, in some embodiments, either the first or second linkages may not be in contact in the contracted configuration.

FIGS. 4C-4D depict the linkages 10, 20 in an expanded, expanded configuration. In some embodiments, the linkages 10, 20 may move between the expanded and contracted configuration with the inflation of the balloon. For example, the linkages may be moved to the expanded configuration when the balloon is inflated, and/or may be moved to the contracted configuration when the balloon is deflated. In some embodiments, the linkages may be moved to the expanded configuration when the balloon is inflated but may be manually manipulated to move toward the contracted configuration.

In some embodiments, at least a portion of either the first linkages 10 or the second linkages 20 may be fixed to the uterine balloon, e.g., to the inner wall surface of the uterine balloon. As the balloon is inflated, the linkages 10 or 20 fixed to the uterine balloon may move outwardly along with the balloon in a radial direction. In some embodiments, the complementary linkages may be driven by the pressure inside the balloon to fill the gaps formed in between the linkages fixed to the balloon. As described previously, pegs 28 of the second linkages 20 may be free to move along the slots 18 of the first linkage 10. In this way, the pegs may allow the second linkages 20 to move relative to the first linkages 10, such that if either the first or second linkages are fixed to the balloon, the relative motion between the complementary linkage and the balloon may still be allowed. For example, in some embodiments, the first linkages 10 are fixed to the uterine balloon while the second linkages 20 are not fixed to the uterine balloon such that relative movement between the balloon and the second linkages 20 can occur. Inflation of the balloon causes the first linkages 10 to move in the radial direction with the balloon. In some embodiments, pressure inside the balloon may drive the second linkages 20 to slide relative to the first linkages 10. During inflation of the balloon, the pegs 28 of the second linkages 20 may slide out of the slots 18 of the first linkages. As a result, the first and second linkages may move from a collapsed, overlapping state to an expanded, side-by-side state. In some embodiments, in the expanded state, the first and second linkages form a ring. The ring may have a larger diameter than the diameter of the patient's cervix. In other embodiments, the arrangement may be reversed such that the second linkages 20 are fixed to the uterine balloon while the first linkages 10 are not fixed to the uterine balloon. Of course, embodiments in which neither linkage is fixed to the balloon, both linkages are fixed to the balloon, as well as embodiments in which at least one linkage body from a set of linkages is fixed to the balloon, are also contemplated. The linkages may be fixed to the balloon in any suitable manner, including, but not limited to, welding, adhesives, and/or any other suitable attachment system.

As shown in FIGS. 4C-4D, in the expanded configuration, the linkages 10, 20 may no longer be overlapping as they were in the contracted configuration of FIGS. 4A-4B. In the expanded configuration, the second linkage 20 may have slid along axial direction A within the slots 18, confined in the transverse direction T by the pegs 28. When the balloon has been sufficiently inflated, such that either first or second linkages move radially outwards from one another, the second linkage 20 may fall into place between neighboring first linkages 10, as shown in FIGS. 4C-4D. The first protrusion 24 may be situated in the first indent 14, and the second protrusion 26 may be situated in the second indent 16 of the first linkage 10. Entry of the protrusions 24, 26 into the corresponding indents 14, 16 of the first linkages 10 may lock the second linkage 20 in place relative to the first linkages 10 to maintain the support in an expanded configuration.

In some embodiments, as shown in the side view of FIG. 4D, the bodies of the linkages 10, 20 may be substantially coplanar in the expanded configuration. This arrangement may occur when the pegs 28 of the linkage 20 slide out of the opening formed in the indent 16, co-linear with the slots 18. In other words, the pegs 28 may only be axially (e.g., along axis A) fixed in the slots 18 along one direction (e.g., towards indent 14), and able to exit the slots in an opposing direction (e.g., towards indent 16). The inflation of the balloon may subsequently urge the linkage 20 in-plane with the first linkage 10, locking the protrusions 24, 26 in indents 14, 16 respectively. It should be appreciated that the engagement of the protrusions and indents may effectively lock the linkages in the axial direction A (at least in the inflation state of the balloon, with a generally fixed radius), preventing the linkages from returning back to the contracted configuration of FIGS. 4A-4B. In this way, at least a portion of the balloon may have a fixed diameter greater than the cervical diameter, such that the balloon may be generally prevented from exiting the uterus through the cervix without unlocking the support from the expanded configuration.

In some embodiments, the support may be unlocked from the expanded configuration when one or more second linkages 20 are pulled out of plane of the first linkages 10. In this way, the pegs 28 of the linkages 20 may re-engage in the slots 18 of the linkages 10, and the support may return to its contracted configuration, as shown in FIGS. 4A-4B.

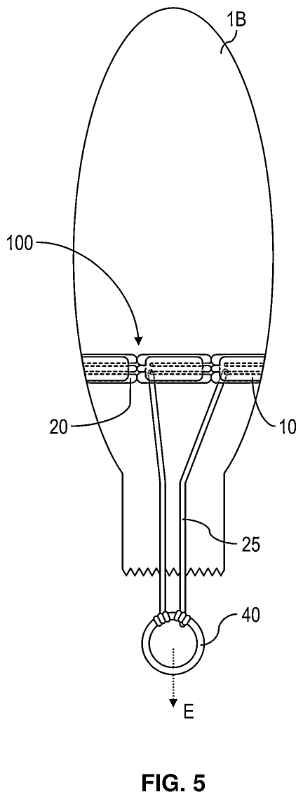

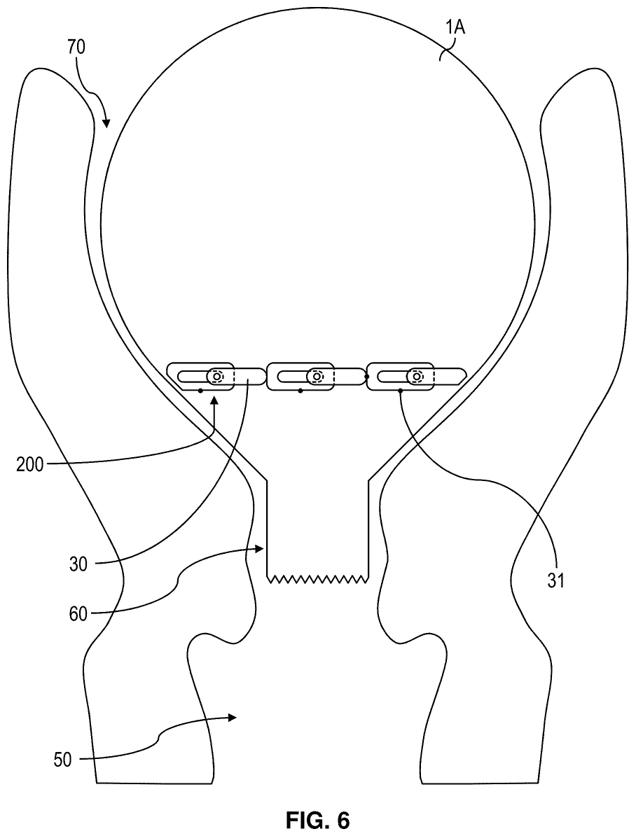

FIG. 5 shows a deflated uterine balloon 1B with a contracted support 100. As shown in the figure, the support 100 may include multiple first linkages 10 overlying multiple second linkages 20. The pegs of the second linkages may be engaged with slots of the first linkages, as depicted in FIGS. 4A-4B. The support 100 may be unlocked from the expanded configuration by a clinician applying a downwardly directed force (e.g., along direction E of FIG. 5) on a pull ring 40. In some embodiments, pulling on the pull ring 40 may contract the support. The pull ring may be connected to the pegs of the second linkage through one or more tethers, such that applying a force to the pull ring 40 (which may be any suitable shape/structure besides a ring) may pull the second linkages out of plane of the first linkages. In this way, the protrusions of the second linkage may be disengaged from the indents of the first linkage, and the pegs may slide along the slots until the linkages are once again at least partially overlapping, as shown in FIG. 5. In this way, the overall size footprint of the support may be reduced, allowing the deflated balloon to be extracted from the uterus. It should be appreciated that in some embodiments, the support 100 may revert to its contracted configuration in response to the deflation of the balloon, without any external input (e.g., a clinician pulling on a pull ring).

FIGS. 6-9 depict another embodiment of a uterine balloon support. FIG. 6 shows an inflated uterine balloon 1A with a support 200 including multiple linkages 30 installed in a patient's uterus 70. As shown in FIG. 6, in the expanded configuration, the support 200 may be positioned in the uterus 70, superior to the cervix 60. Each linkage may be fixed to an internal face of the balloon at least at one location, such as location 31, as shown in FIG. 6. The linkage may be fixed to the balloon with any suitable means, including, but not limited to welding, adhesives, and/or any other suitable attachment mechanism. As noted with reference to FIGS. 2-5, the support 200 may help maintain the balloon within the uterus 70 relative to the cervix 60.

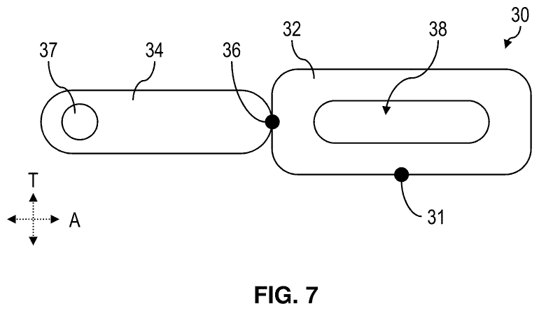

FIG. 7 shows a close view of a linkage 30 used in a support. In some embodiments, the linkage 30 may include at least a first body portion 32 and a second body portion 34. The two bodies 32, 34 may be fixed to one another at least at one location, such as location 36. The bodies may be fixed to one another with any suitable means, and/or, in some embodiments, may be formed together, such that the location 36 represents a connection between the bodies rather than an affixing point. The first body 32 may include a slot 38 extending along an axial direction A. The slot 38 may be confined entirely within the body 32. In some embodiments, the slot 38 may extend through the body 32 along an in-plane direction of the figure.

The body 34 of the linkage 30 may include a protrusion 37. The protrusion 37 may extend out of plane of the substantially flat body 34, akin to the pegs 28 of FIG. 3B. The protrusion 37 may be sized to slide along a slot 38 of a neighboring linkage along the axial direction A. Accordingly, the slot 38 may be located co-linear with the protrusion 37. The slot 38 may substantially reduce movement of the protrusion 37 along the transverse direction T.

It should be appreciated that although FIGS. 6-7 depict the linkage 30 being fixed to the balloon at a location on the first body 32 (e.g., location 31), the linkage 30 may be fixed to the balloon at any suitable location or combination of locations on body 32 and/or body 34.

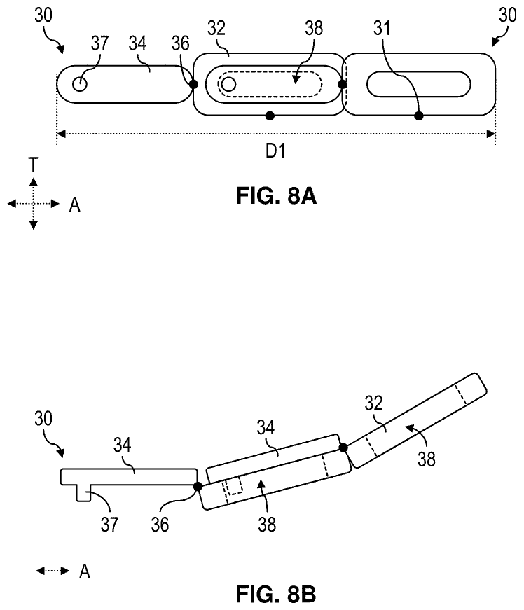

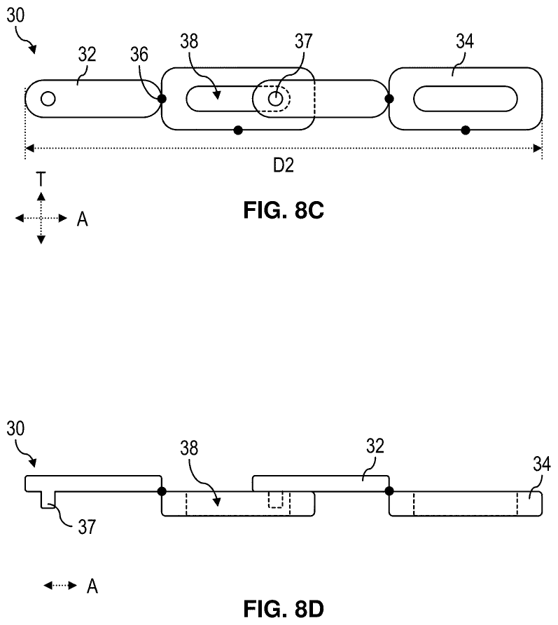

FIGS. 8A-8D depict a process of contracting the support according to some embodiments. It should be appreciated that FIGS. 8A-8D depict a partial view of the support (e.g., only a pair of linkages), and that any suitable number of linkages may be employed for the support, as the present disclosure is not so limited.

FIG. 8A shows a pair of linkages 30 engaged with one another in a front view, and FIG. 8B shows the same pair of linkages 30 from a top view. As shown in FIGS. 8A-8B, the linkages 30 may be engaged with one another when a protrusion 37 of one linkage is positioned in the slot 38 of another linkage. As such, the linkages may move in an axial direction A for at least the length of the slot 38. In other words, movement of neighboring linkages with respect to one another may be limited by the length of the slot 38 along the axial direction A.

As shown in FIG. 8B, the linkages 30 may be curved with respect to the axial direction A. This may be due to the differences in axial length of the bodies 32, 34. In some embodiments, as shown in FIG. 8B, the body 32 may be longer along the axial direction A than the body 34. Of course, embodiments in which the body 34 is the same dimension or longer than the body 32 along direction A are also contemplated. In the contracted configuration, the pair of engaged linkages may have an axial length D1 measured along the direction A, as shown in FIG. 8A.

In order to expand the support, a clinician may inflate the associated uterine balloon, which may increase its radial dimension. Accordingly, the linkages, which may be fixed to the balloon, may move along with the balloon walls. As noted previously, neighboring linkages may be permitted to move axially a distance corresponding to the slot 38 length along direction A. As such, as the balloon expands, neighboring linkages may move apart from one another while maintaining the engagement of the protrusions 37 and slots 38.

FIGS. 8C-8D depict a pair of linkages in an expanded configuration. The linkages 30 may span over a length D2 along the axial direction A. It should be appreciated that the expanded length D2 may be greater than the contracted length D1 by the length of the slots 38. As such, the linkages may be prevented from moving further along the axial direction A, effectively locking the support in the expanded configuration. This configuration may prevent the balloon from falling out of the uterus. As noted previously, the overall size of the locked support may be greater than the cervical opening, such that the support may not comfortably pass the cervix when in the expanded configuration.

It should be appreciated that the support depicted in FIGS. 6-8 may be expanded and contracted with the balloon, due to the connection of the linkages with the interior surface of the balloon.

In some embodiments, as shown in the top view in FIG. 8D, the pair of linkages 30 may be more aligned along the axial direction A, as compared to the contracted configuration of FIG. 8B. It should be appreciated that the support may be in a ring-like shape in its expanded configuration, and so the pair of linkages shown in FIG. 8D (and similarly, in FIG. 4D) may exhibit some curvature with respect to the direction A, such that they may form part of a ring shape.

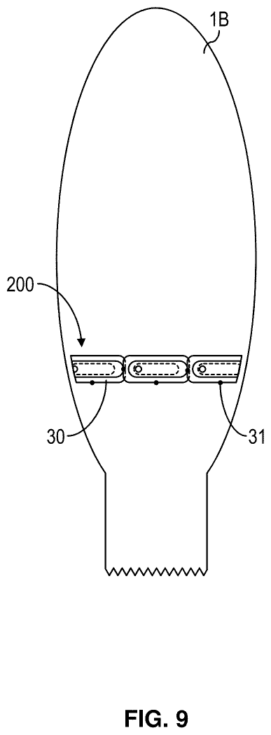

FIG. 9 shows a support 200 in an expanded configuration, positioned inside a deflated balloon 1B. In some embodiments, the support may automatically contract when the balloon is deflated, such that the distance between linkages 30 fixed to the balloon (e.g., at locations 31) may be reduced along with the deflating balloon's radius. The neighboring linkages 30 may therefore overlap one another (similar to FIG. 8A) to accommodate the shrinkage of the balloon. The overlapping of neighboring linkages may sufficiently reduce the footprint of the support to allow it, along with the deflated balloon, to be extracted from the patient's uterus without significant discomfort.

In some embodiments, the support 100 of FIGS. 2-5 and support 200 of FIGS. 6-9 form a generally ring-shaped structure in their expanded configuration. It should be appreciated that the present disclosure is not limited by the two-dimensional nature of the ring-like support. In some embodiments, a balloon may include linkages distributed along its internal surface corresponding with a three-dimensional support. In this way, the balloon shape, as well as its position, may be maintained by the support. In some embodiments, a support may be three-dimensional.

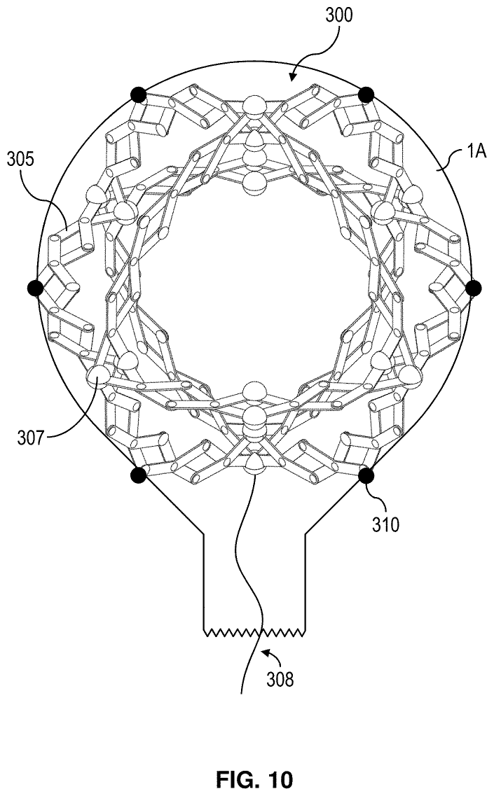

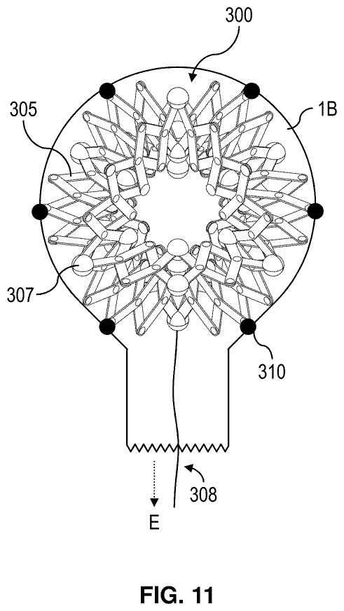

FIGS. 10-11 depict an illustrative embodiment of a three-dimensional support 300. The three-dimensional support may be expanded and/or contracted along with the balloon, and may serve the function of helping to maintain the balloon in place (at least relative to the cervix) in its expanded state. In some embodiments, the three-dimensional support 300 may include a plurality of linkages 305 connected to one another at hinged junctions 307. In some embodiments, as shown in FIGS. 10-11, the support 300 may expand/collapse in an isotropic manner. However, embodiments in which a three-dimensional support expands/collapses in an anisotropic manner are also contemplated. In some embodiments, such as with the illustrative embodiment shown in FIGS. 10-11, a three-dimensional support 300 may be formed of an auxetic structure (e.g., a Hoberman sphere) at least partially embedded in the uterine balloon.

In some embodiments, the support 300 may be controlled by the inflation/deflation of the balloon akin to supports 100, 200 previously described, to eliminate the need for a clinician to have to manually and/or directly expand or contract the support. The three-dimensional support 300 may be connected to the balloon at one or more locations 310, such that the support 300 may expand/collapse along with the inflation (see inflated balloon 1A in FIG. 10) and deflation (see deflated balloon 1B in FIG. 11) of the balloon. In some embodiments, the support 300 may include a tether 308, which may function similarly to the pull ring of FIGS. 2-5, such that it may allow the clinician to extract the balloon from the patient. In some embodiments, the tether 308 (which may include a pull ring and/or any other suitable structure to allow the clinician to comfortably handle the tether) may serve as a handle to collapse the support 300. In these embodiments, the tether 308 may be connected to the support 300, such that a downwardly directed force, (e.g., along direction E as shown by the arrow of FIG. 11), may contract the support 300, and allow the clinician to remove the deflated balloon 1B.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

1. A medical device comprising:
a balloon configured to be positioned at least partially in a uterus of a patient; and
a support disposed inside the balloon,
wherein the support has a contracted configuration and an expanded configuration, the support being configured to expand from the contracted configuration to the expanded configuration due to inflation of the balloon,
and wherein in the contracted configuration, the support permits movement of the balloon through a cervix of the patient, and wherein in the expanded configuration, the support inhibits movement of the balloon through the cervix.

2. The medical device of claim 1, wherein inflation of the balloon reversibly locks the support in the expanded configuration.

3. The medical device of claim 1, wherein deflation of the balloon causes the support to contract from the expanded configuration toward the contracted configuration.

4. The medical device of claim 1, further comprising a tether connected to the support, wherein the tether is configured to be manipulated to cause the support to contract from the expanded configuration toward the contracted configuration.

5. The medical device of claim 1, wherein the support comprises a plurality of linkages.

6. The medical device of claim 5, wherein the plurality of linkages includes a first plurality of linkages and a second plurality of linkages, wherein each of the first plurality of linkages comprises at least one slot extending along an axial direction of the first plurality of linkages, wherein each of the second plurality of linkages comprises at least one projection extending normal to the axial direction, and wherein the at least one projection of each of the second plurality of linkages is configured to be partially axially moveable relative to the at least one slot of the first plurality of linkages.

7. The medical device of claim 6, further comprising at least one tether connected to at least one projection of at least one of the second plurality of linkages, wherein the at least one tether is configured to be manipulated to contract the support from the expanded configuration toward the contracted configuration.

8. The medical device of claim 5, wherein at least one of the plurality of linkages is fixed to the balloon at least at one location.

9. The medical device of claim 5, wherein each of the plurality of linkages includes at least one slot extending partially along an axial direction of the linkage, wherein each of the plurality of linkages includes at least one projection extending out of plane of the linkage, and wherein the at least one projection is configured to be partially axially moveable relative to the at least one slot of a neighboring linkage.

10. The medical device of claim 1, further comprising at least one hemostatic covering on an external surface of the balloon.

11. The medical device of claim 10, wherein the at least one hemostatic covering comprises an absorbent material pre-loaded with a hemostatic agent.

12. The medical device of claim 1, further comprising a pump configured to inflate the balloon.

13. The medical device of claim 1, further comprising at least one electrode configured to electrostimulate the uterus.

14. A method of operating a medical device comprising:
inserting a balloon at least partially in a uterus of a patient;
inflating the balloon to apply pressure to the uterus; and
expanding a support disposed inside the balloon from a contracted configuration to an expanded configuration,
wherein in the expanded configuration, the support is larger than a cervix of the patient, and wherein the support, in the expanded configuration, is positioned in the uterus, superior to the cervix of the patient.

15. The method of claim 14, wherein inflating the balloon causes the support to expand from the contracted configuration to the expanded configuration.

16. The method of claim 14, further comprising:
deflating the balloon;
contracting the support disposed inside the balloon from the expanded configuration toward the contracted configuration to permit movement of the support through the cervix; and
removing the balloon and the support from the uterus of the patient.

17-18. (canceled)

19. The method of claim 14, wherein the support comprises a plurality of linkages.

20. The method of claim 19, wherein the plurality of linkages includes a first plurality of linkages and a second plurality of linkages, wherein each of the first plurality of linkages comprises at least one slot extending along an axial direction of the first plurality of linkages, wherein each of the second plurality of linkages comprises at least one projection extending normal to the axial direction, and wherein the at least one projection of each of the second plurality of linkages is configured to be partially axially moveable relative to the at least one slot of the first plurality of linkages.

21. (canceled)

22. The method of claim 19, wherein at least one of the plurality of linkages is fixed to the balloon at least at one location.

23. The method of claim 19, wherein each of the plurality of linkages includes at least one slot extending partially along an axial direction of the linkage, wherein each of the plurality of linkages includes at least one projection extending out of plane of the linkage, and wherein the at least one projection is configured to be partially axially moveable relative to the at least one slot of a neighboring linkage.

24-27. (canceled)
